(19) United States
(12) Patent Application Publication
(10) Pub. No.: US 2026/0345543 A1
(43) Pub. Date: Nov. 5, 2026

(54) Bridged Bicyclic Compounds and their Derivatives as Antiepileptic Agents and Methods of Use Thereof

(71) Applicants: Florida Atlantic University Board of Trustees, Boca Raton, FL (US); Nova Southeastern University, Fort Lauderdale, FL (US)

(72) Inventors: Salvatore Lepore, Delray Beach, FL (US); Samantha E. Stilley, Tequesta, FL (US); Krishna Yadavalli, Lafayette, IN (US); Kenneth Dawson-Scully, Boca Raton, FL (US)

(21) Appl. No.: 19/104,884

(22) PCT Filed: Jun. 15, 2023
(86) PCT No.: PCT/US23/25444

§ 371 Date: Feb. 19, 2025

Related U.S. Application Data
(60) Provisional application No. 63/399,312, filed on Aug. 19, 2022.

(51) Int. Cl. A61K 31/216; A61P 25/08; C07C 69/013

(52) U.S. Cl. CPC A61K 31/216; A61P 25/08; C07C 69/013; C07C 2602/44

(57) ABSTRACT

Methods of preventing or treating seizures and treating epilepsy in an individual include the administration of bridged bicyclic small molecule compounds. These compounds exhibit an antiepileptic function in the individual when administered in a therapeutically effective dose. Use of the methods and antiepileptic compounds provides protection from seizures at low and higher doses of compound without any toxic effects.

### CROSS-REFERENCE TO RELATED APPLICATIONS

The present application claims priority under 35 U.S.C. § 119(e) to the U.S. Provisional Patent Application No. 63/399,312, filed Aug. 19, 2022. The entire disclosure of the application noted above is incorporated herein by reference.

### STATEMENT REGARDING FEDERALLY SPONSORED RESEARCH

This invention was made with government support under grant number GM110651 awarded by the National Institutes of Health. The government has certain rights in the invention.

### FIELD OF THE INVENTION

The invention relates generally to the fields of pharmacology, medicine, and neurology. In particular, the invention relates to methods of treating or preventing seizures and treating epilepsy by administering compounds exhibiting antiepileptic effects.

### BACKGROUND

Epilepsy is a prevalent disease in the United States, with about 1.2% of the population being affected. Epilepsy can be diagnosed following two or more seizures that occur oftentimes when there is increased neuronal excitability that causes disturbances and muscle convulsions, and in some, loss of consciousness. Although the occurrence is high, few new antiepileptic drugs (AEDs) have been FDA-approved in recent years and one-third of patients find themselves without sufficient treatment. Existing drugs (e.g., sodium valproate, valproic acid, phenytoin, levetiracetam) remain effective, but they are often accompanied by side-effects. Recent literature suggests that 30% of adults with new-onset epilepsy have AED-resistant seizures (Galanopoulou et al., Epilepsia, 2012, 53 (3), 571-582; Dalic et al., Neuropsychiatric Disease and Treatment, 2016, 12, 2605; Mohanraj R. and Brodie M. J., Seizure, 2005, 14 (5), 318-323). Additionally, patients that were once treatable subsequently develop resistance to AEDs, further emphasizing the need for the development of more effective therapies (Löscher et al., 2020, Pharmacol. Rev., 72, 606-638). Therefore, there is an urgent need for new and potent AEDs, especially those from underrepresented compound classes that may offer alternatives to patients who remain refractory to existing drugs. The majority of existing FDA-approved AEDs possess heterocyclic scaffolds with varying degrees of unsaturation. More recent efforts over the past decade have not revealed a significant change from this heterocyclic motif (Özbek, O.; Gürdere, M. B., Med. Chem. Res. 2020, 29, 1553-1578).

### SUMMARY

Described herein are methods of preventing or treating seizures and treating epilepsy in an individual. The methods include administration of compounds exhibiting antiepileptic effects to the individual in a therapeutically effective amount to exhibit antiepileptic function in the individual. These antiepileptic compounds contain a mostly saturated all-carbon bridged bicyclic scaffold, specifically, a highly ‘three-dimensional’ core scaffold and the presence of five contiguous chiral centers. In the experiments described below, using the methods and antiepileptic compounds described herein, protection from seizures was observed at low and higher concentrations of compound without evidence of a toxic effect. The in vivo experiments, using the C. elegans assay, described below provide evidence that an epileptic attack can be prevented.

Accordingly, described herein is a method of preventing or treating seizures and treating epilepsy in an individual including administering to the individual a compound of Formula 1:

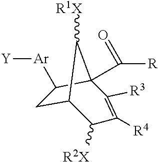

or an enantiomer, diastereomer, racemic mixture, or a pharmaceutically acceptable salt or solvate thereof, wherein:
Ar is aryl;
Y is an ortho, meta, or para aryl substituent selected from the group consisting of alkyl, alkyloxy, alkylamino, NR⁵R⁶ and halo;
X is O, S, or NH bonded to R¹ or R²;
R is H, alkyl, aryl, OH, alkyloxy, aryloxy, NH₂, alkylamino, NR⁵R⁶, or arylamino;
R¹ and R² are alkylcarbonyl, arylcarbonyl, alkyl, or H, individually;
R³ is arylCH═CH, alkylCH═CH, or alkyl;
R⁴ is H, alkyl, or aryl; and
R⁵ and R⁶ are alkyl, individually.

In the method, the compound is administered in a therapeutically effective amount to exhibit antiepileptic function in the individual. The therapeutically effective amount is a dose of about 0.001 mg/kg to about 10 mg/kg (e.g., about 0.001, 0.005, 0.01, 0.05, 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.1 mg/kg). In a typical embodiment, the individual is a human, but can be any animal (e.g., mammals such as rodents, canines, cats, bovines, non-human primates, etc.). In embodiments, administering the compound to the individual alleviates epileptic symptoms and decrease duration of seizures without evidence of a toxic effect. In embodiments, administering the compound to the individual decreases time to recovery when the individual experiences a seizure. The compound can exhibit antiepileptic function in the presence of a pro-convulsant. In the methods, typically the compound is within a composition also including a pharmaceutically acceptable carrier. Typically, the compound is administered to the individual prior to onset of a seizure.

In some embodiments of the antiepileptic compound, alkyl is a saturated hydrocarbon moiety containing up to six carbons; and, aryl is a 5- or 6-membered aryl or heteroaryl group. In some embodiments, the alkyl is an alkyloxy, alkylamino, alkylCH═CH, or alkylcarbonyl. In one embodiment, the compound has the formula:

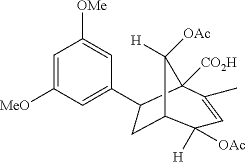 [SMILES: COc1cc(OC)cc(C2CC3C(OC(C)=O)C=C(C)C2(C(=O)O)C3OC(C)=O)c1]

or an enantiomer, or a pharmaceutically acceptable salt thereof.

In another embodiment, the compound has the formula:

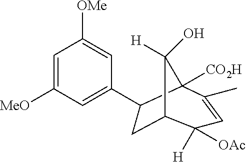 [SMILES: COc1cc(OC)cc(C2CC3C(OC(C)=O)C=C(C)C2(C(=O)O)C3O)c1]

or an enantiomer, or a pharmaceutically acceptable salt thereof.

In another embodiment, the compound has the formula:

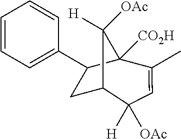 [SMILES: CC(=O)OC1C=C(C)C2(C(=O)O)C(c3ccccc3)CC1C2OC(C)=O]

or an enantiomer, or a pharmaceutically acceptable salt thereof.

Also described herein is a method of preventing or treating seizures and treating epilepsy in an individual including administering to the individual a compound of Formula 2:

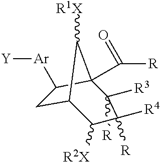

or an enantiomer, diastereomer, racemic mixture, analogue, derivative or a pharmaceutically acceptable salt or solvate thereof, wherein:
Ar is aryl;
Y is an ortho, meta, or para aryl substituent selected from the group consisting of alkyl, alkyloxy, alkylamino, NR⁵R⁶ and halo;
X is O, S, or NH bonded to R¹ or R²;
R is H, alkyl, aryl, OH, alkyloxy, aryloxy, NH₂, alkylamino, NR⁵R⁶, or arylamino;
R¹ and R² are alkylcarbonyl, arylcarbonyl, alkyl, or H, individually;
R³ is arylCH═CH, alkylCH═CH, or alkyl;
R⁴ is H, alkyl, or aryl; and
R⁵ and R⁶ are alkyl, individually.

In such an embodiment of the method, the compound can have the formula:

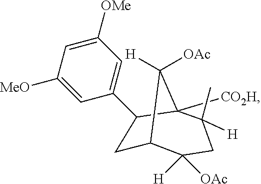 [SMILES: COc1cc(OC)cc(C2CC3C(OC(C)=O)CC(C)C2(C(=O)O)C3OC(C)=O)c1]

or an enantiomer, diastereomer, racemic mixture, analogue or derivative or a pharmaceutically acceptable salt thereof.

The terms “group,” “functional group,” “pendant group,” “moiety,” “molecular moiety,” or the like are somewhat synonymous in the chemical arts and are used to refer to distinct, definable portions or units of a molecule, and to units that perform some function. Examples of functional groups that are suitable for the compounds described herein include, but are not limited to, aryl or heteroaryl group, alkyoxy, alkylamino, alkylthio, dialkylamino, halo, hydroxy, amino, thiol, arylamino, alkanoyl, arylcarbonyl, arylvinyl, alkylvinyl, or the like.

As used herein, the term “aryl” refers to aromatic hydrocarbons such as phenyl and naphthyl that may be substituted with a variety of functional and/or alkyl groups (e.g., C1-C6 alkyl).

As used herein, the term “heteroaryl” refers to aromatic cycles where one or more heteroatoms form part of the ring. The heteroaryl ring may also be substituted with a variety of functional and/or alkyl groups (e.g., C1-C6 alkyl).

As used herein, the term “alkyl” refers to a saturated hydrocarbon fragment. For example, in one embodiment, an alkyl can be a saturated hydrocarbon moiety containing up to six carbons (e.g., methyl, ethyl, propyl, isopropyl).

As used herein, the term “alkyoxy” refers to a group comprised of an alkyl group connected to an oxygen (e.g., methoxy, ethyoxy)

As used herein, the term “alkylamino” refers to a group comprised of an alkyl group connected to a trivalent nitrogen (e.g., methylamino, ethylamino).

As used herein, the term “dialkylamino” refers to a group comprised of two alkyl groups (though not necessarily the same) connected to a trivalent nitrogen (e.g., dimethylamino, diethylamino, ethylmethylamino).

As used herein, the term “arylamino” refers to an aryl group connected to a trivalent nitrogen.

As used herein, the term “alkanoyl” refers an alkyl group connected to a carbonyl.

As used herein, the term “arylcarbonyl” refers an aryl group connected to a carbonyl.

As used herein, the term “arylvinyl” refers to an aryl group connected to an ethylene group (e.g., phenylvinyl, also abbreviated as C₆H₅CHCH).

As used herein, the term “alkylvinyl” refers to an alkyl group connected to an ethylene group (e.g., methylvinyl, also abbreviated as CH₃CHCH).

As used herein, the term “halo” or “halogen” refers to F, Cl, Br, or I.

As used herein, when referring to a compound, the terms “biologically active compound” and “bioactive compound” mean a compound having a physiological or biological effect on animals or humans or cells therefrom.

By the term “antiepileptic compound” is meant any compound that exhibits antiepileptic function. Typically, an antiepileptic compound is capable of treating (alleviating, mitigating) epilepsy (e.g., protection from seizures) in an individual. In some embodiments, an antiepileptic compound is a compound of Formula 1:

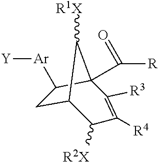

or a pharmaceutically acceptable salt or solvate thereof, wherein: Ar=aryl; Y=aryl substituent (ortho, meta, or para) selected from the group consisting of: alkyl, alkyloxy, alkylamino, R⁵R⁶N, and halo; X═O, N, or S; R═H, alkyl, aryl, OH, alkyloxy, aryloxy, NH₂, alkylamino, R⁵R⁶N, or arylamino; R¹ and R²=alkylcarbonyl, arylcarbonyl, alkyl, or H, individually (R¹ and R² can be the same group, or they can be different groups); R³=arylCH═CH, alkylCH═CH, alkyl; R⁴═H, alkyl, or aryl; and R⁵ and R⁶=alkyl, individually (R⁵ and R⁶ can be the same group, or they can be different groups).

In other embodiments, an antiepileptic compound is a compound of Formula 2:

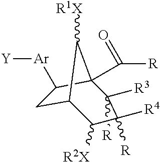

or an enantiomer, diastereomer, racemic mixture, or a pharmaceutically acceptable salt or solvate thereof, wherein: Ar is aryl; Y is an ortho, meta, or para aryl substituent selected from the group consisting of alkyl, alkyloxy, alkylamino, NR⁵R⁶ and halo; X is O, S, or NH bonded to R¹ or R²; R is H, alkyl, aryl, OH, alkyloxy, aryloxy, NH₂, alkylamino, NR⁵R⁶, or arylamino; R¹ and R² are alkylcarbonyl, arylcarbonyl, alkyl, or H, individually; R³ is arylCH═CH, alkylCH═CH, or alkyl; R⁴ is H, alkyl, or aryl; and R⁵ and R⁶ are alkyl, individually.

As depicted in FIG. 1, the terms “resveramorph” and “RVM” refer to scaffolds that are modified forms of resveratrol natural products that contain a significant amount of three-dimensional character, specifically, a bridged bicycle. As described in Example 1 below, some resveramorphs, including RVM-3, exhibit antiepileptic properties.

The term “purified” means separated from many other entities (small molecules, compounds, proteins, nucleic acids), and does not require the material to be present in a form exhibiting absolute purity, exclusive of the presence of other entities. In some embodiments, a small molecule, compound, protein, nucleic acid or other entity is considered pure (purified) when it is removed from substantially all other entities.

By the terms “to modulate” and “modulates” is meant to increase or decrease. These terms can refer to increasing or decreasing an activity, level or function of a molecule (e.g., protein, peptide, nucleic acid, small molecule, metabolite), or effecting a change with respect to one or more biological or physiological mechanisms, effects, responses, functions, pathways or activities in which, for example, seizures are involved.

The terms “patient,” “subject” and “individual” are used interchangeably herein, and mean a subject, typically a mammal, to be treated, diagnosed, and/or to obtain a biological sample from. Subjects include, but are not limited to, humans, non-human primates, horses, cows, sheep, pigs, rats, mice, insects, dogs, and cats. A human in need of epilepsy or seizure treatment is an example of a subject.

The terms “agent” and “therapeutic agent” as used herein refer to a chemical entity or biological product, or combination of chemical entities or biological products, administered to a subject (a mammal such as a human) to treat a disease or condition (e.g., epilepsy, seizures). Examples of therapeutic agents include small molecules (compounds) and biologics, which may be referred to herein as a “drug” or “therapeutic drug”.

As used herein, the terms “therapeutic treatment” and “therapy” are defined as the application or administration of a therapeutic agent (e.g., an antiepileptic compound as described herein or composition including an antiepileptic compound) or therapeutic agents to a patient who has a disease, a symptom of disease or a predisposition toward a disease, with the purpose to cure, heal, alleviate, relieve, alter, remedy, ameliorate, improve or affect the disease, the symptoms of disease, or the predisposition toward disease.

Although methods, compounds and compositions similar or equivalent to those described herein can be used in the practice or testing of the present invention, suitable methods, compounds and compositions are described below. All publications, patent applications, and patents mentioned herein are incorporated by reference in their entirety. In the case of conflict, the present specification, including definitions, will control. The particular embodiments discussed below are illustrative only and not intended to be limiting.

### BRIEF DESCRIPTION OF THE DRAWINGS

FIG. 1 shows the structure of several RVM compounds evaluated for antiseizure activity.

FIG. 2 is a graph showing recovery time (seconds) for N2 worms (vertical axis) in the presence of M9 or pentylenetetrazol (PTZ) (first two bars). In other experiments, PTZ is combined with either an RVM or sodium valproate.

FIG. 3 left is the structure of RVM-3, and FIG. 3 right is a graph showing recovery time (seconds) for N2 worms (vertical axis) in the presence of M9 or PTZ (first two bars). In other experiments, PTZ is combined with RVM-3 at varying concentrations or sodium valproate.

FIG. 4 is a schematic of a C. elegans electroshock assay.

### DETAILED DESCRIPTION

Described herein are methods of preventing or treating seizures and treating epilepsy in an individual. The methods include administering to the individual an antiepileptic compound of Formula 1 or Formula 2, or a composition containing an antiepileptic compound of Formula I or Formula 2, such that the antiepileptic compound is in a therapeutically effective amount to exhibit antiepileptic function in the individual. In the C. elegans electroshock experiments described below, antiepileptic compounds RVM-3, RVM-6, RVM-11, and RVM-12 restored function to C. elegans in the presence of a seizure promoting agent and RVM-3 treatment of animals in the absence of a seizure promoting agent resulted in normal recovery time. In these experiments, treatment with the antiepileptic compounds was not associated with any toxic effect. The experimental results show that the antiepileptic compounds (RVMs) alleviate symptoms and duration of seizure in an in vivo model.

In embodiments of the methods, to an individual in need thereof (e.g., an individual experiencing a seizure, an individual susceptible to experiencing a seizure, an individual with epilepsy), is administered a compound of Formula 1:

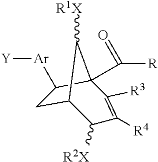

or an enantiomer, diastereomer, racemic mixture or a pharmaceutically acceptable salt or solvate thereof, wherein:
Ar is aryl;
Y is an ortho, meta, or para aryl substituent selected from the group consisting of alkyl, alkyloxy, alkylamino, NR⁵R⁶ and halo;
X is O, S, or NH bonded to R¹ or R²;
R is H, alkyl, aryl, OH, alkyloxy, aryloxy, NH₂, alkylamino, NR⁵R⁶, or arylamino;
R¹ and R² are alkylcarbonyl, arylcarbonyl, alkyl, or H, individually;
R³ is arylCH═CH, alkylCH═CH, or alkyl;
R⁴ is H, alkyl, or aryl; and
R⁵ and R⁶ are alkyl, individually.

In other embodiments of the methods, to an individual in need thereof (e.g., an individual experiencing a seizure, an individual susceptible to experiencing a seizure, an individual with epilepsy), is administered a compound of Formula 2:

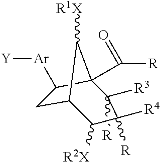

or an enantiomer, diastereomer, racemic mixture, or a pharmaceutically acceptable salt or solvate thereof, wherein:
Ar is aryl;
Y is an ortho, meta, or para aryl substituent selected from the group consisting of alkyl, alkyloxy, alkylamino, NR⁵R⁶ and halo;
X is O, S, or NH bonded to R¹ or R²;
R is H, alkyl, aryl, OH, alkyloxy, aryloxy, NH₂, alkylamino, NR⁵R⁶, or arylamino;
R¹ and R² are alkylcarbonyl, arylcarbonyl, alkyl, or H, individually;
R³ is arylCH═CH, alkylCH═CH, or alkyl;
R⁴ is H, alkyl, or aryl; and
R⁵ and R⁶ are alkyl, individually.

The compound is administered in a therapeutically effective amount to exhibit antiepileptic function in the individual, typically at a dose of about 0.001 mg/kg to about 10 mg/kg. In the methods, administering the compound to the individual can alleviate epileptic symptoms and decrease duration of seizures without evidence of a toxic effect. As described in Example 1, the antiepileptic compounds exhibit antiepileptic function in the presence of a pro-convulsant. Also in the methods, administering the compound to the individual can decrease time to recovery when the individual experiences a seizure. An antiepileptic compound can be administered to the individual at one or more time points (e.g., a first time point and a second time point), including prior to onset of a seizure, at onset, or during a seizure.

The compounds described herein may exist as different diastereomeric isomers than those exemplified both as racemates as well as non-racemic or enantiopure forms. Analogues and derivatives of the compounds described herein are also encompassed by the invention.

In one embodiment of an antiepileptic compound according to Formula 1 or Formula 2, an alkyl is a saturated hydrocarbon moiety containing up to six carbons, and an aryl is a 5- or 6-membered aryl or heteroaryl group. Any suitable alkyl can be used. Examples of alkyl-containing substituents that can be used in the antiepileptic compounds described herein include alkyloxy, alkylamino, alkylCH═CH, and alkylcarbonyl. Similarly, any suitable aryl can be used. Examples of aryl-containing substituents that can be used in the compounds described herein include arylcarbonyl, arylamino, and arylvinyl. When referring to an aryl substituent, it may be, for example, one that is ortho, meta and/or para substituted.

The antiepileptic compounds according to Formula 1 used in the methods described herein, and their synthesis, are described in U.S. Pat. No. 10,759,735, which is incorporated herein by reference in its entirety. Novel antiepileptic compounds according to Formula 2 can be synthesized from Formula 1, which is itself obtained using previously reported methods (e.g., U.S. Pat. No. 10,759,735). The conversion of Formula 1 to Formula 2 can be achieved by an alkene addition reaction (e.g., hydrogenation). The antiepileptic compounds can be synthesized using any suitable methods.

In one embodiment of an antiepileptic compound according to Formula 1, the compound has the formula (and its enantiomer):

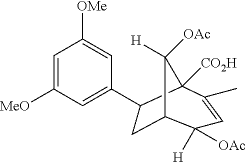 [SMILES: COc1cc(OC)cc(C2CC3C(OC(C)=O)C=C(C)C2(C(=O)O)C3OC(C)=O)c1]

In another embodiment of an antiepileptic compound according to Formula 1, the compound has the formula (and its enantiomer):

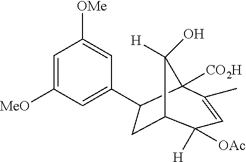 [SMILES: COc1cc(OC)cc(C2CC3C(OC(C)=O)C=C(C)C2(C(=O)O)C3O)c1]

In another embodiment of an antiepileptic compound according to Formula 1, the compound has the formula (and its enantiomer):

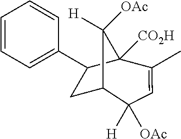 [SMILES: CC(=O)OC1C=C(C)C2(C(=O)O)C(c3ccccc3)CC1C2OC(C)=O]

In one embodiment of an antiepileptic compound according to Formula 2, the compound has the formula (and its enantiomer)

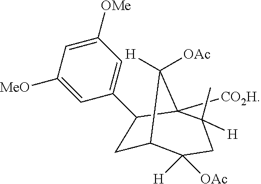 [SMILES: COc1cc(OC)cc(C2CC3C(OC(C)=O)CC(C)C2(C(=O)O)C3OC(C)=O)c1]

In some embodiments, an antiepileptic compound as described herein encompasses any other combinations of the structural features described herein.

Compositions including an antiepileptic compound according to any embodiments described herein typically also include a pharmaceutically acceptable carrier.

In some embodiments, the individual is suffering from a seizure or seizures or is susceptible to suffering from a seizure(s). The antiepileptic compounds and compositions described herein may be used to treat, reduce or prevent any type of seizure, e.g., seizures caused by anything that interrupts the normal connections between neurons in the brain. Examples of pathologies, in addition to epilepsy, that cause seizures include drug and alcohol withdrawal, fever, stroke, meningitis, etc.

Any suitable methods of administering an antiepileptic compound or composition as described herein to an individual may be used. In these methods, the compounds and compositions can be administered to an individual by any suitable route, e.g., oral, buccal (e.g., sub-lingual), parenteral (e.g., subcutaneous, intramuscular, intradermal, or intravenous), and topical (i.e., both skin and mucosal surfaces, including airway surfaces), administration. In an embodiment, an antiepileptic compound or composition may be administered systemically by intravenous injection. In another embodiment, an antiepileptic compound or composition may be administered directly to a target site, by, for example, surgical delivery to an internal or external target site (e.g., brain) or by catheter to a site accessible by a blood vessel. If administered via intravenous injection, the compound or composition may be administered in a single bolus, multiple injections, or by continuous infusion (e.g., intravenously, by peritoneal dialysis, pump infusion). For parenteral administration, the compound or composition is preferably formulated in a sterilized pyrogen-free form.

As indicated above, an antiepileptic compound or composition as described herein may be in a form suitable for sterile injection. To prepare such a composition, the suitable active therapeutic agent(s) (e.g., a therapeutically effective amount of an antiepileptic compound) is dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution (D5W, 0.9% sterile saline). The aqueous formulation may also contain one or more preservatives (e.g., methyl, ethyl or n-propyl p-hydroxybenzoate). In cases where the therapeutic agent(s) (1 or more antiepileptic compounds) is only sparingly or slightly soluble in water, a dissolution enhancing or solubilizing agent can be added, or the solvent may include 10-60% w/w of propylene glycol or the like. The compounds and compositions described herein may be administered to an individual (e.g., rodents, humans, nonhuman primates, canines, felines, ovines, bovines, insects) in any suitable formulation according to conventional pharmaceutical practice (see, e.g., Remington: The Science and Practice of Pharmacy (21st ed.), ed. A. R. Gennaro, Lippincott Williams & Wilkins, (2005) and Encyclopedia of Pharmaceutical Technology, (3^{rd} ed.) eds. J. Swarbrick and J. C. Boylan, Marcel Dekker, CRC Press, New York (2006), a standard text in this field, and in USP/NF). A description of exemplary pharmaceutically acceptable carriers and diluents, as well as pharmaceutical formulations, can be found in Remington: supra. Other substances may be added to the compounds and compositions to stabilize and/or preserve them.

The therapeutic methods described herein in general include administration of a therapeutically effective amount of the antiepileptic compounds and compositions described herein to an individual (e.g., human) in need thereof, particularly a human. Such treatment will be suitably administered to individuals, particularly humans, suffering from, having, susceptible to, or at risk for a disease, disorder, or symptom thereof (e.g., epilepsy, seizures). Determination of those individuals “at risk” can be made by any objective or subjective determination by a diagnostic test or opinion of a subject or health care provider. The methods described herein can include measuring antiepileptic effects of an antiepileptic compound. In these methods, any suitable protocol and/or model can be used to measure the antiepileptic effects of a compound, including the C. elegans electroshock model described in Example 1.

### Effective Doses

The antiepileptic compounds and compositions described herein are preferably administered to an individual in need thereof (e.g., human with epilepsy) in an effective amount, that is, an amount capable of producing a desirable result in a treated individual. Desirable results include but are not limited to one or more of the following: restoring function (neurological, behavioral), achieving normal recovery time, preventing a seizure(s), etc. Such a therapeutically effective amount can be determined according to standard methods. Toxicity and therapeutic efficacy of the antiepileptic compounds and compositions utilized in the methods described herein can be determined by standard pharmaceutical procedures. As is well known in the medical and veterinary arts, dosage for any one individual depends on many factors, including the individual's size, body surface area, age, the particular composition to be administered, time and route of administration, general health, and other drugs being administered concurrently. A delivery dose of an antiepileptic composition as described herein is typically between about 0.001 mg/kg of antiepileptic compound to about 10 mg/kg (e.g., 0.001, 0.002, 0.003, 0.004, 0.005, 0.006, 0.007, 0.008, 0.009, 0.01, 0.02, 0.03, 0.04, 0.05, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.1 mg/kg) of antiepileptic compound and can be determined based on preclinical efficacy and safety. A dosage amount for an individual may be based on achieving drug concentrations at the cellular level required for the cells involved to achieve their normal function (i.e., cellular homeostasis). The experiments described herein with worms (C. elegans) involved immersing the animals in drug solution. Even at very dilute concentrations (e.g., 10 picomolar), an antiseizure effect can be clearly observed with RVM-3. This effect is at least 10,000-fold more potent than drugs such as valproate and levetiracetam. From these results, a dosing range in animals of 0.001 mg/kg to about 10 mg/kg was estimated. The usual starting dose of levetiracetam is 10 mg/kg. Levetiracetam is considered to be among the more potent antiepileptic agents.

### EXAMPLES

The present invention is further illustrated by the following specific example. The example is provided for illustration only and should not be construed as limiting the scope of the invention in any way.

### Example 1—Bridged Bicyclic Compounds and their Derivatives as Antiepileptic Agents

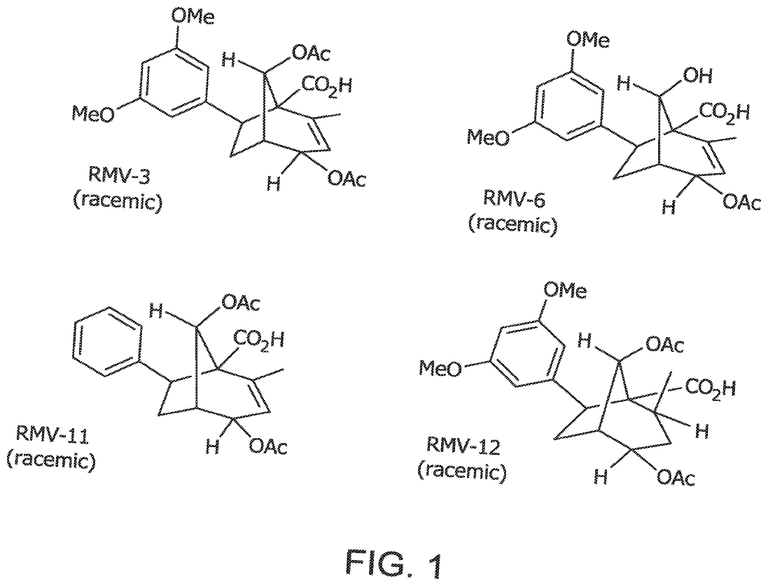

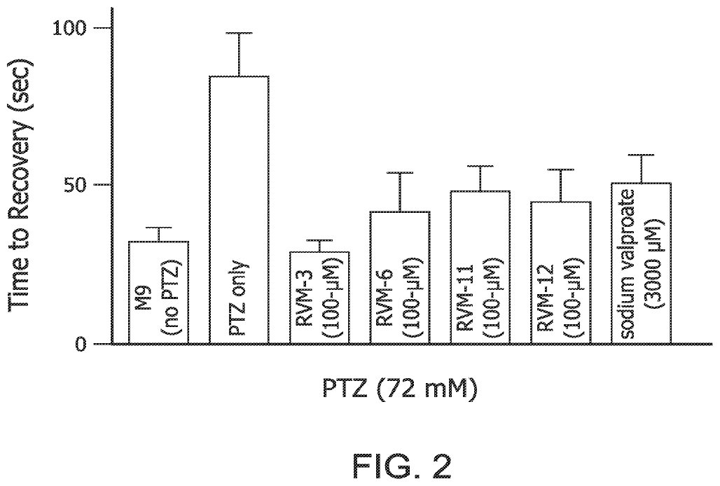

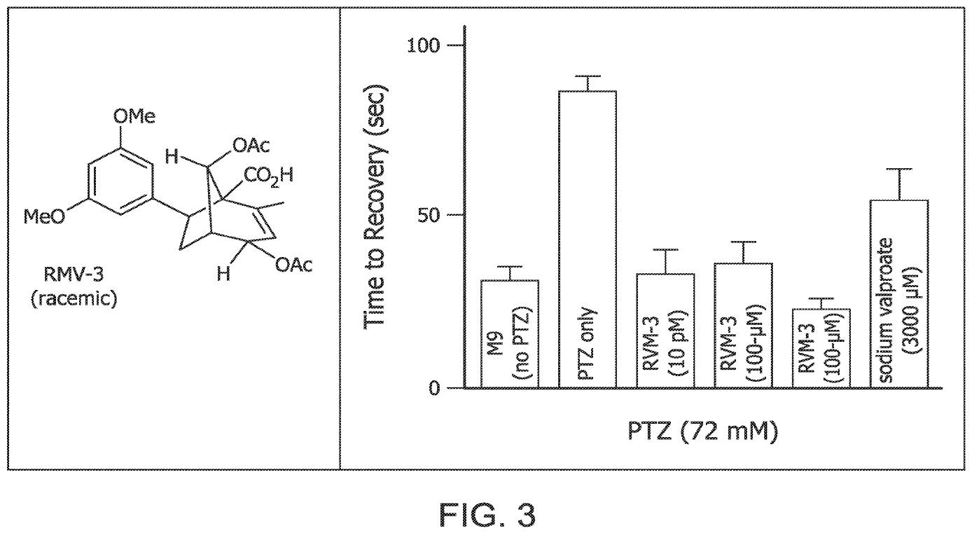

Compounds known as resveramorphs (RVMs) (FIG. 1) are used in the methods described herein as highly potent antiepileptic agents as measured by the C. elegans electroshock experiment (see below). The structures of these RVMs are comprised of an all-carbon bridged bicyclic scaffold that is fully saturated, a motif that is without precedent in the AED literature. At a concentration of 100 μM, RVM-3, RVM-6, RVM-11, and RVM-12 restored function to C. elegans in the presence of PTZ, a seizure promoting agent (FIG. 2). Further evaluation of RVM-3 revealed that normal recovery time was achieved at 10 pM relative to C. elegans animals not pretreated with PTZ (FIG. 3). Protection was also observed at the significantly higher concentration of 100 μM concentration without evidence of a toxic effect. Sodium valproate, an FDA approved AED that was used as a control in these experiments, exhibited a relatively modest restoration of recovery time even at a concentration of 3 mM (3000 μM). As seen in these data, RVMs alleviate symptoms and duration of seizure. It is expected that a pharmacological intervention based on RVMs will provide relief from epileptic events at low dosages.

The C. elegans seizure assay includes placing L4 C. elegans into a small tube that is capped on both sides by copper wires. A shock of 47 V for three seconds is then administered. The worms demonstrate a stiff paralysis state and uncoordinated rigid unilateral body bends. This suggests muscle convulsions as seen in high temperature seizures. Recovery is recorded when the animals present with coordinated three sinusoidal movements, which is then characterized as normal swimming activity. The use of Pentylenetetrazole (PTZ), a GABAA receptor antagonist, enhances the seizure-like phenotypes seen in C. elegans. When applied, PTZ prevents the binding of GABA on this receptor, and, since GABA is an inhibitory neurotransmitter, this creates a large excitatory signal, thus causing seizure-like activity in the animal (Thapliyal, S.; Babu, K, Bio-protocol, 2018, 8 (17), e2989). Previous work demonstrates that when this method is used with loss of function mutants, unc-25 and unc-49, significantly more time is needed for recovery when compared to wild type. Unc-25 encodes the GABA biosynthetic enzyme GAD and unc-49 encodes the GABAA receptor. This decreases the inhibitory signal to the muscles leading to the results observed here. Importantly, when these animals are additionally treated with PTZ, sensitivity to electroshock is increased. However, when the mutant animals are treated with antiepileptic drugs such as Retigabine, Keppra (levetiracetam), and sodium valproate, increased sensitivity is rescued. Therefore, this method, is a proven model for screening AEDs.

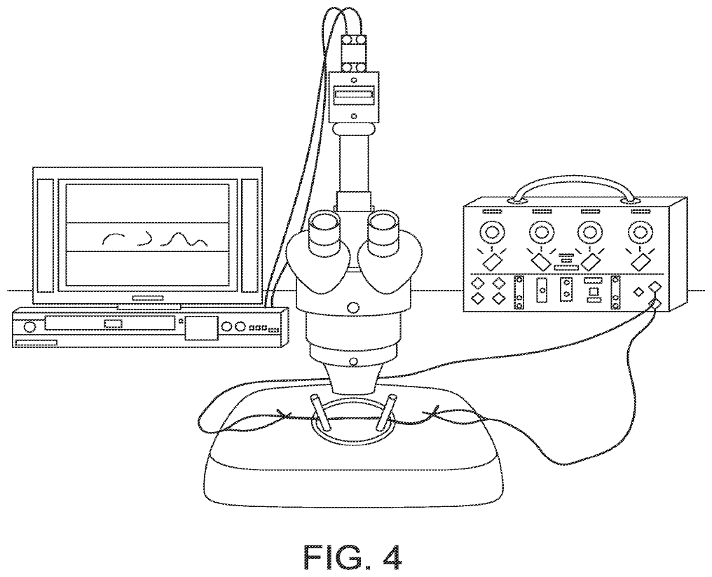

This C. elegans assay offers a new model for the evaluation of electroconvulsive seizures (FIG. 4). There are few previous reports that describe seizure susceptibility in worms (Pandey et al., Seizure 2010, 19 (7), 439-442) and none that examine electroconvulsive seizures. This assay allows for quick and efficient evaluation of the antiepileptic efficacy of compounds. In the assay, approximately 10 worms are inserted into a clear tube containing an M9 saline solution. Copper wire attached to a square pulse generating stimulator plugs each side of the tube, allowing for 1 cm between electrodes. The M9 saline solution allows for an even conductance of current. For trials involving RVM and PTZ (72 mM), the test compound is added directly to the M9, and worms are allowed to incubate for 30 minutes in the RVM solution before testing. Upon an electroshock of 47 V for 3 seconds, the worms demonstrate a stiff paralysis state and rigid unilateral body bends. This suggests muscle convulsions as seen in high temperature seizures. Recovery is recorded when the animals present with three sinusoidal movements, which is then characterized as normal swimming activity. These analyses are accomplished under a microscope with a camera and attached recording device, allowing for further subsequent evaluation. With this seizure model, a decrease in time to recovery was observed when animals were treated with various concentrations of RVMs.

### OTHER EMBODIMENTS

Any improvement may be made in part or all of the antiepileptic compounds, compositions, and method steps. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents (e.g., analogues and derivatives) of the subject matter recited in the claims appended hereto as permitted by applicable law. Moreover, any combination of the above-described elements in all possible variations thereof is encompassed by the invention unless otherwise indicated herein or otherwise clearly contraindicated by context.

What is claimed is:

1. A method of preventing or treating seizures and treating epilepsy in an individual comprising administering to the individual a compound of Formula 1: 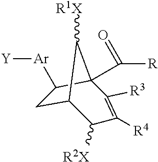
or an enantiomer, diastereomer, racemic mixture, or a pharmaceutically acceptable salt or solvate thereof, wherein:
Ar is aryl;
Y is an ortho, meta, or para aryl substituent selected from the group consisting of alkyl, alkyloxy, alkylamino, NR⁵R⁶ and halo;
X is O, S, or NH bonded to R¹ or R²;
R is H, alkyl, aryl, OH, alkyloxy, aryloxy, NH₂, alkylamino, NR⁵R⁶, or arylamino;
R¹ and R² are alkylcarbonyl, arylcarbonyl, alkyl, or H, individually;
R³ is arylCH═CH, alkylCH═CH, or alkyl;
R⁴ is H, alkyl, or aryl; and
R⁵ and R⁶ are alkyl, individually,
wherein the compound is administered in a therapeutically effective amount to exhibit antiepileptic function in the individual.

2. The method of claim 1, wherein the therapeutically effective amount is a dose of about 0.001 mg/kg to about 10 mg/kg.

3. The method of claim 1, wherein the compound has the formula: 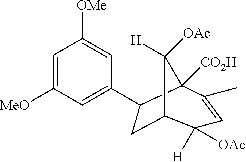 [SMILES: COc1cc(OC)cc(C2CC3C(OC(C)=O)C=C(C)C2(C(=O)O)C3OC(C)=O)c1]
or an enantiomer, or a pharmaceutically acceptable salt thereof.

4. The method of claim 1, wherein administering the compound to the individual alleviates epileptic symptoms and decrease duration of seizures without evidence of a toxic effect.

5. The method of 1, wherein the compound exhibits antiepileptic function in the presence of a pro-convulsant.

6. The method of claim 1, wherein administering the compound to the individual decreases time to recovery when the individual experiences a seizure.

7. The method of claim 1, comprising administering the compound to the individual prior to onset of a seizure.

8. The method of claim 1, wherein the compound is comprised within a composition comprising a pharmaceutically acceptable carrier.

9. The method of claim 1, wherein the individual is a mammal.

10. A method of preventing or treating seizures and treating epilepsy in an individual comprising administering to the individual a compound of Formula 2: 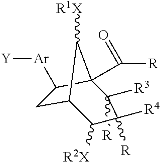
or an enantiomer, diastereomer, racemic mixture or a pharmaceutically acceptable salt or solvate thereof, wherein:
Ar is aryl;
Y is an ortho, meta, or para aryl substituent selected from the group consisting of alkyl, alkyloxy, alkylamino, NR⁵R⁶ and halo;
X is O, S, or NH bonded to R¹ or R²;
R is H, alkyl, aryl, OH, alkyloxy, aryloxy, NH₂, alkylamino, NR⁵R⁶, or arylamino;
R¹ and R² are alkylcarbonyl, arylcarbonyl, alkyl, or H, individually;
R³ is arylCH═CH, alkylCH═CH, or alkyl;
R⁴ is H, alkyl, or aryl; and
R⁵ and R⁶ are alkyl, individually,
wherein the compound is administered in a therapeutically effective amount to exhibit antiepileptic function in the individual.

11. A method of preventing or treating seizures and treating epilepsy in an individual comprising administering to the individual a compound having the formula: 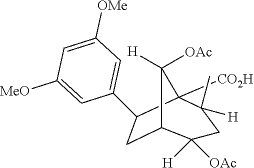 [SMILES: COc1cc(OC)cc(C2CC3C(OC(C)=O)CC(C)C2(C(=O)O)C3OC(C)=O)c1]
or an enantiomer thereof,
wherein the compound is administered in a therapeutically effective amount to exhibit antiepileptic function in the individual.

12. A compound of Formula 2: 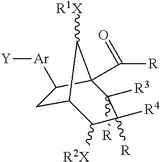
or an enantiomer, diastereomer, racemic mixture, or a pharmaceutically acceptable salt or solvate thereof, wherein:
Ar is aryl;
Y is an ortho, meta, or para aryl substituent selected from the group consisting of alkyl, alkyloxy, alkylamino, NR⁵R⁶ and halo;
X is O, S, or NH bonded to R¹ or R²;
R is H, alkyl, aryl, OH, alkyloxy, aryloxy, NH₂, alkylamino, NR⁵R⁶, or arylamino;
R¹ and R² are alkylcarbonyl, arylcarbonyl, alkyl, or H, individually;
R³ is arylCH═CH, alkylCH═CH, or alkyl;
R⁴ is H, alkyl, or aryl; and
R⁵ and R⁶ are alkyl, individually.

13. The compound of claim 12, having the formula:

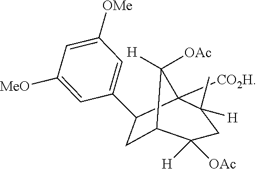 [SMILES: COc1cc(OC)cc(C2CC3C(OC(C)=O)CC(C)C2(C(=O)O)C3OC(C)=O)c1]

14. A composition comprising the compound of claim 12 and a pharmaceutically acceptable carrier.

15. The composition of claim 14, wherein the compound has the formula: 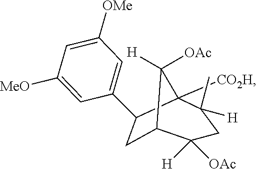 [SMILES: COc1cc(OC)cc(C2CC3C(OC(C)=O)CC(C)C2(C(=O)O)C3OC(C)=O)c1]
or is an analogue or derivative thereof.
